(19) United States
(12) Patent Application Publication
(10) Pub. No.: US 2016/0220744 A1
(43) Pub. Date: Aug. 4, 2016

(54) COLOSTRUM COLLECTION SYSTEM

(71) Applicant: NEOMED, INC., Woodstock, GA (US)

(72) Inventors: Benjamin M. DAVIS, Woodstock, GA (US); Mariann CARY, Canton, GA (US); Aaron N. INGRAM, Canton, GA (US); Mark Martin COSTELLO, Co. Mayo (IE); Adrian McDERMOTT, Co. Galway (IE)

(73) Assignee: NEOMED, INC., Woodstock, GA (US)

(21) Appl. No.: 15/014,569

(22) Filed: Feb. 3, 2016

Related U.S. Application Data
(60) Provisional application No. 62/111,493, filed on Feb. 3, 2015.
(60) Provisional application No. 62/169,738, filed on Jun. 2, 2015.

(51) Int. Cl. A61M 1/06; A61M 39/20; A61M 1/00

(52) U.S. Cl. CPC A61M 1/062; A61M 1/007; A61M 39/20

(57) ABSTRACT

A system and method for collection of colostrum or other enteral fluid using a breast pump or other collection device directly into a syringe for storage and dispensing of the colostrum for feeding a newborn infant. A collar adaptor couples the syringe body or barrel in sealing engagement with the discharge coupling of the breast pump to transfer expressed colostrum directly from the breast pump to the collection syringe.

### CROSS-REFERENCE TO RELATED APPLICATION

This application claims the priority benefit of U.S. Provisional Patent Application Ser. No. 62/111,493 filed Feb. 3, 2015 and U.S. Provisional Patent Application Ser. No. 62/169,738 filed Jun. 2, 2015, which are hereby incorporated by reference herein.

### TECHNICAL FIELD

The present invention relates generally to the field of neonatal feeding, and more particularly to a system and method for collection, storage and delivery of colostrum.

### BACKGROUND

Colostrum, a milky fluid produced by the mammary glands of humans and other mammals shortly before and/or shortly after birth, has been found to contain antibodies and concentrated nutrients such as proteins, carbohydrates, fats, vitamins, minerals and other materials beneficial to neonates or newborns. As such, collection and feeding of colostrum to newborns is often desirable.

Collection and delivery of colostrum, however, has proved challenging. Colostrum often comes in very slowly and in very small quantities. Known methods for collection of colostrum include manual expression and mechanical expression using a pump system. Known pump expression systems include both open and closed systems. In open systems the pump mechanism is exposed to the collected colostrum. Open system pumps typically incorporate a sealed system in order to maintain suction. In closed systems, a barrier prevents the collected colostrum from traveling to the pump system. Closed system pumps benefit from a ventilated system and typically do not require seals.

Breast pump systems generally have a coupling or receiver for attachment of bottles or other collection containers. This coupling site is usually configured as a conventional collar having a downward depending rim with an internal set of screw threads. Collection containers such as bottles used with pump systems typically have a rim with corresponding external screw threads to removably couple the collection container with the breast pump.

Collection using previously known methods (both manual expression and pump expression) typically uses different containers for collecting colostrum and administering said colostrum to an infant, necessitating a transfer between containers. Due to colostrum's high viscosity, colostrum commonly sticks to the containers causing excessive waste. Transfer between containers also increases the risk of contamination or spillage.

Therefore, needs exist for improved systems and methods for collection and delivery of colostrum. These systems and methods would ideally collect and deliver colostrum efficiently, so that there was little waste of collected colostrum. It would also be beneficial if these improved systems and methods were compatible with a variety of commercially available breast pumps.

### SUMMARY

In example embodiments, the present invention provides improved systems and methods for collection, storage and delivery of colostrum for feeding to neonates or newborns.

In one aspect, the present invention relates to a system for collection of colostrum. The system preferably includes a breast pump for expressing colostrum from a mammalian breast, the breast pump having a breast pump discharge port for delivery of the expressed colostrum. The system preferably also includes a collection syringe body including an internal chamber, a proximal end having a flange and a plunger opening in fluid communication with the internal chamber, and a distal end having a syringe discharge port in fluid communication with the internal chamber. The system preferably also includes a collar adaptor including a receiver for releasable engagement of the proximal end of the syringe body and a coupling for attachment to the breast pump discharge port.

In another aspect, the invention relates to a collar adaptor for a colostrum collection system. The collar adaptor preferably includes a receiver for releasable engagement of a proximal end of a syringe body and a coupling for attachment to a breast pump discharge port.

In still another aspect, the invention relates to a method of collecting colostrum from a mammalian breast into a containment syringe. The method preferably includes coupling a collection syringe body to a breast pump with a collar adaptor, the collection syringe body having an internal chamber for receiving and containing collected colostrum, a proximal end having a flange and a plunger opening in fluid communication with the internal chamber, and a distal end having a syringe discharge port in fluid communication with the internal chamber; the breast pump having a breast pump discharge port; and the collar adaptor having a receiver for releasable engagement of the proximal end of the syringe body and a coupling for attachment to the breast pump discharge port, The method preferably further includes expressing colostrum from the mammalian breast by operation of the breast pump, and discharging the expressed colostrum from the breast pump discharge port directly to the internal chamber of the collection syringe body through the plunger opening.

In another aspect, the invention relates to a system including a syringe for receiving collected colostrum directly from a breast pump or other collection means, and a coupling collar that holds the flange of the syringe in place and connects to the collection/bottle coupling part of the breast pump.

In another aspect, the invention relates to a method of collection of colostrum from a breast pump or other collection means into a syringe, using the above-described collection system.

In still another aspect, the invention relates to an adaptor coupling or conversion kit for adapting an existing breast pump for use in connection with collection of colostrum into a syringe-based containment.

These and other aspects, features and advantages of example embodiments of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 shows a colostrum collection system according to an example embodiment of the present invention.

FIG. 2 shows a colostrum collection system according to another example embodiment of the present invention.

FIG. 3 is a detailed view of a syringe and collar adaptor for attachment of the syringe to a breast pump.

FIG. 4 is another detailed view of collar adaptor and syringe for attachment of the syringe to a breast pump.

FIG. 5 is another detailed view of a collar adaptor and syringe for attachment of the syringe to a breast pump.

FIG. 6 shows a colostrum collection system according to another example embodiment of the present invention.

FIG. 7 shows a collar adaptor according to another example embodiment of the same invention.

FIG. 8 is an exploded view of the collar adaptor of FIG. 7 with a syringe.

FIG. 9 is an exploded view the collar adaptor and syringe of FIG. 8 with a breast pump.

FIG. 10 shows an exploded view of the collar adaptor in FIG. 7, depicting the removable gasket.

FIG. 11 shows an exploded view of a colostrum collection system according to another example embodiment of the same invention.

FIG. 12 shows a collar adaptor and syringe according to another example embodiment of the present invention.

FIG. 13 is an exploded side view of the collar adaptor and syringe of FIG. 12.

FIG. 14 is a detailed view of the collar adaptor of FIG. 12.

FIG. 15 is a bottom view of the collar adapter in FIG. 12.

FIG. 16 shows a collar adaptor and syringe according to another example embodiment of the present invention.

FIG. 17 is a side view of the syringe and collar adaptor of FIG. 16.

FIG. 18 shows a collar adaptor, syringe and breast pump according to another example embodiment of the present invention.

FIG. 19 is an exploded view of the colostrum collection system of FIG. 18.

FIG. 20 shows a collar adaptor and syringe according to another example embodiment of the present invention.

FIG. 21 is a side view of the collar adaptor and syringe of FIG. 20 coupled together.

FIG. 22 shows a collar adaptor and syringe according to another example embodiment of the present invention.

FIG. 23 is a perspective view of the collar adaptor and syringe of FIG. 22.

FIG. 24 shows a collar adaptor and syringe according to another example embodiment of the present invention.

FIG. 25 is a bottom view of the collar adaptor and syringe of FIG. 24.

FIG. 26 is a side view of the collar adaptor and syringe of FIG. 24.

FIG. 27 shows a collar adaptor and gasket according to another example embodiment of the present invention.

FIG. 28 is a side view of the collar adaptor and gasket of FIG. 27.

FIG. 29 is an alternate side view of the collar adaptor and gasket of FIG. 27.

FIG. 30 shows a syringe with the collar adaptor and gasket of FIG. 27.

FIG. 31 shows the collar adaptor and syringe of FIG. 30 attached to an example configuration of a breast pump to create a colostrum collection system.

FIG. 32 is a detailed view of the colostrum collection system of FIG. 31.

FIG. 33 is a detailed view of the collar adaptor and syringe of FIG. 31.

FIG. 34 shows the collar adaptor and syringe of FIG. 30 attached to another example configuration of a breast pump to create a colostrum collection system.

FIG. 35 is a detailed view of the colostrum collection system of FIG. 34.

FIG. 36 is a detailed view of the collar adaptor and syringe of the colostrum collection system of FIG. 34.

FIG. 37 is a bottom view of the collar adaptor and syringe of FIG. 36.

FIG. 38 shows a collar adaptor and syringe according to another example embodiment of the present invention.

FIG. 39 is a detailed view of the collar adaptor of FIG. 38.

FIG. 40 shows a collar adaptor and syringe according to another example embodiment of the present invention.

FIG. 41 is a top view of the collar adaptor of FIG. 40.

FIG. 42 is a detailed view of the collar adaptor of FIG. 40.

FIG. 43 is a top view of the collar adaptor and syringe of FIG. 40.

FIG. 44 shows male thread dimensions of a collar adaptor according to an example embodiment of the present invention.

FIG. 45 shows female thread dimensions of an example breast pump coupling to which a collar adaptor according to an example embodiment of the present invention may be attached.

### DETAILED DESCRIPTION OF EXAMPLE EMBODIMENTS

The present invention may be understood more readily by reference to the following detailed description of example embodiments of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

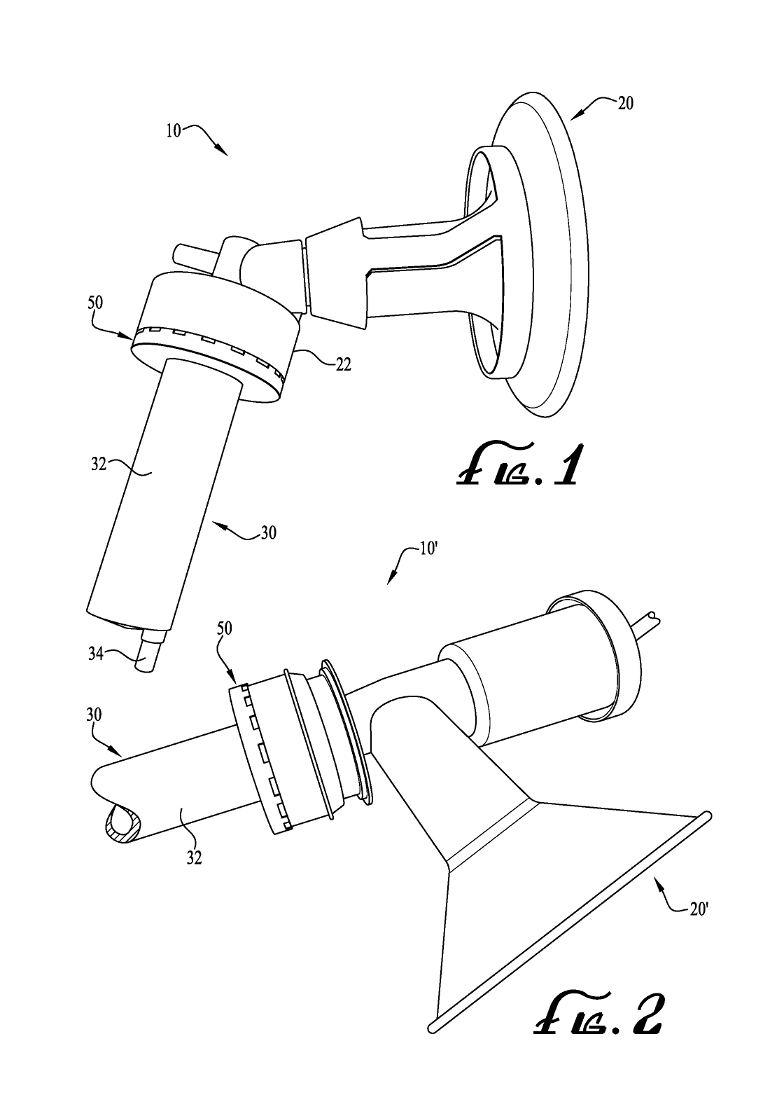

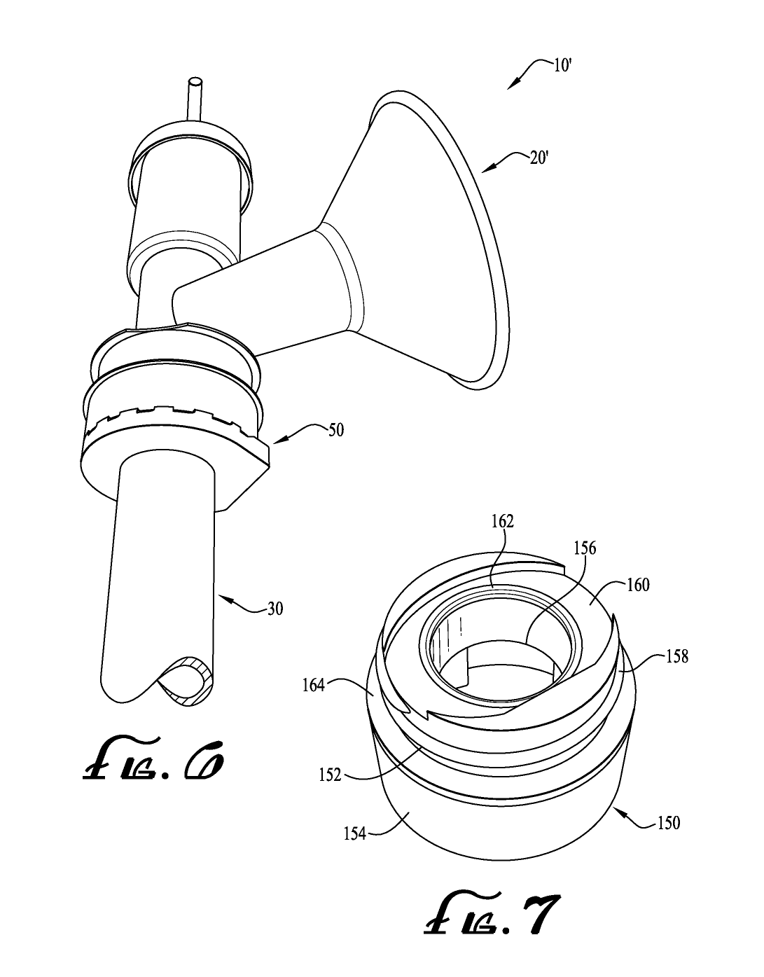

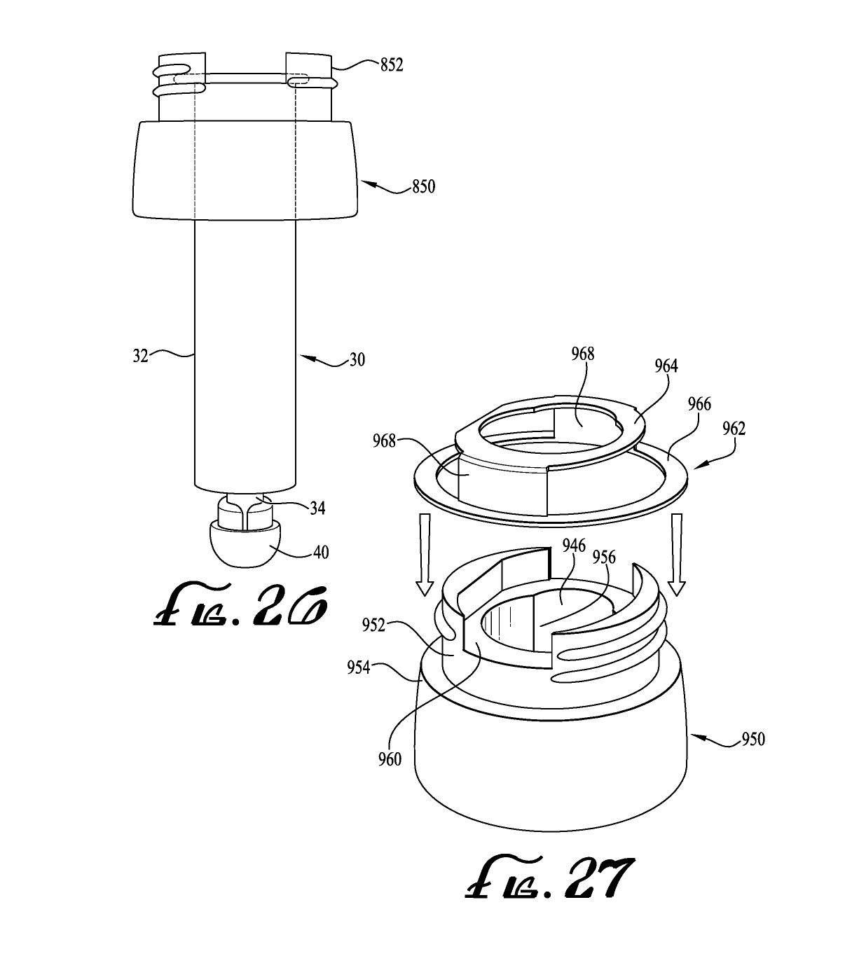

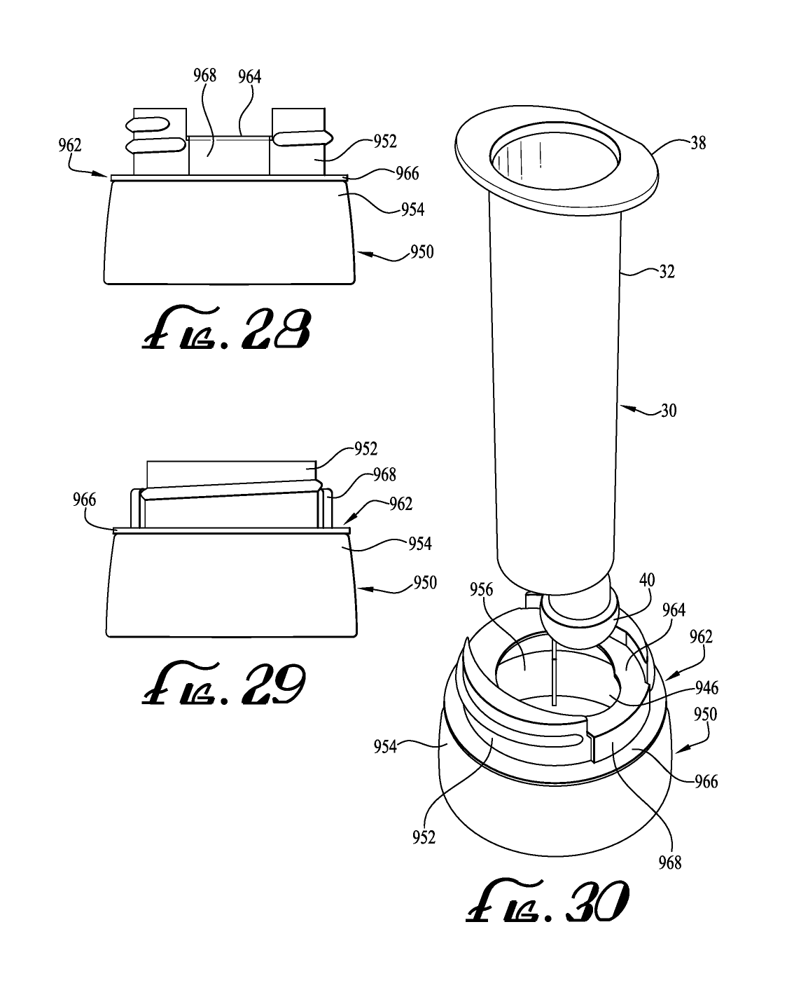

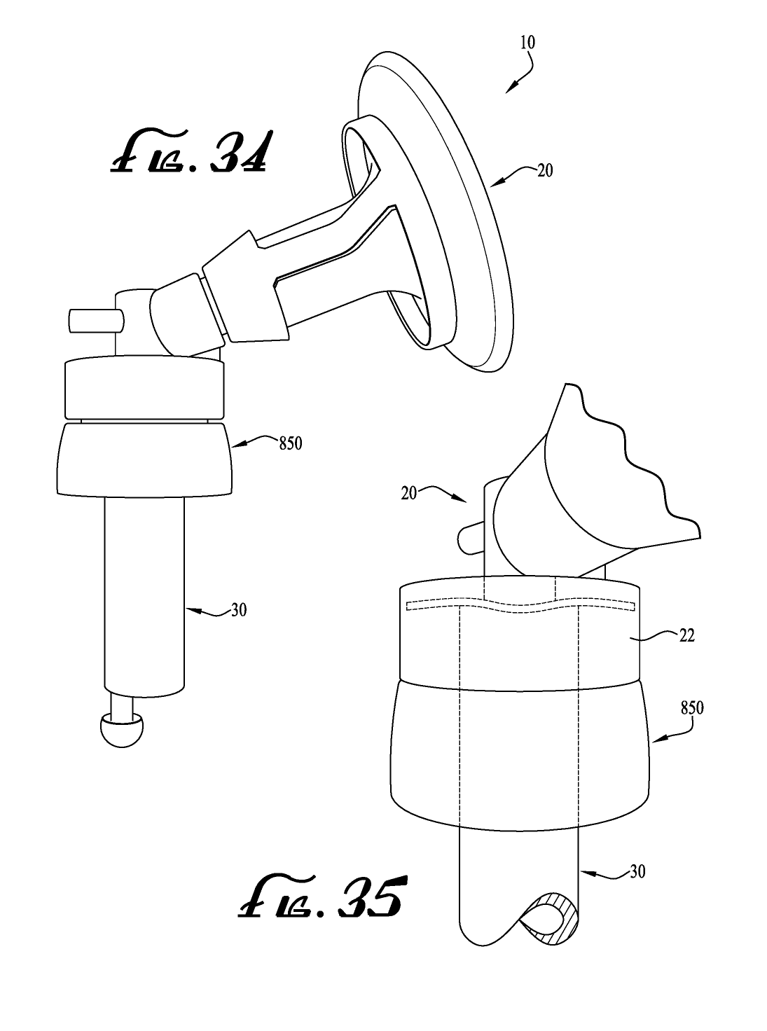

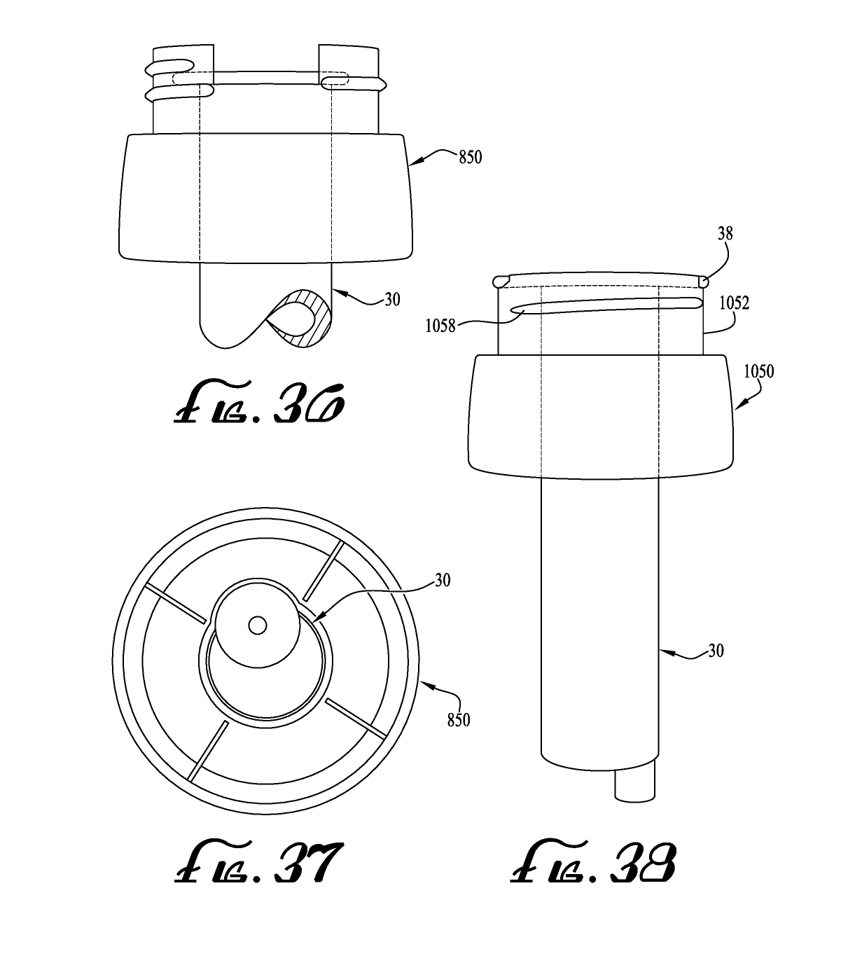

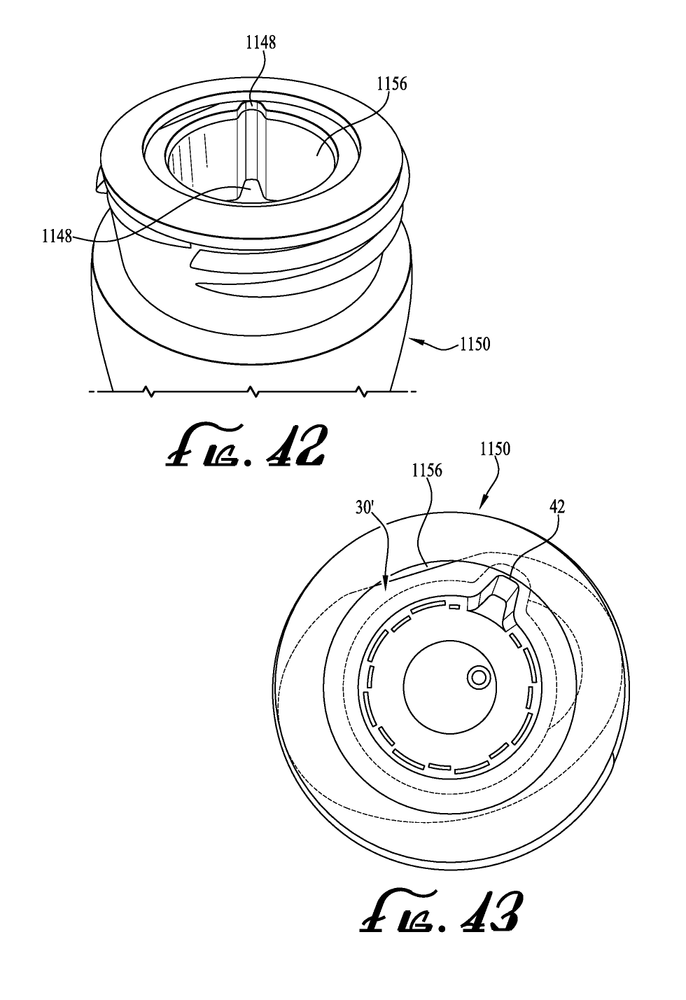

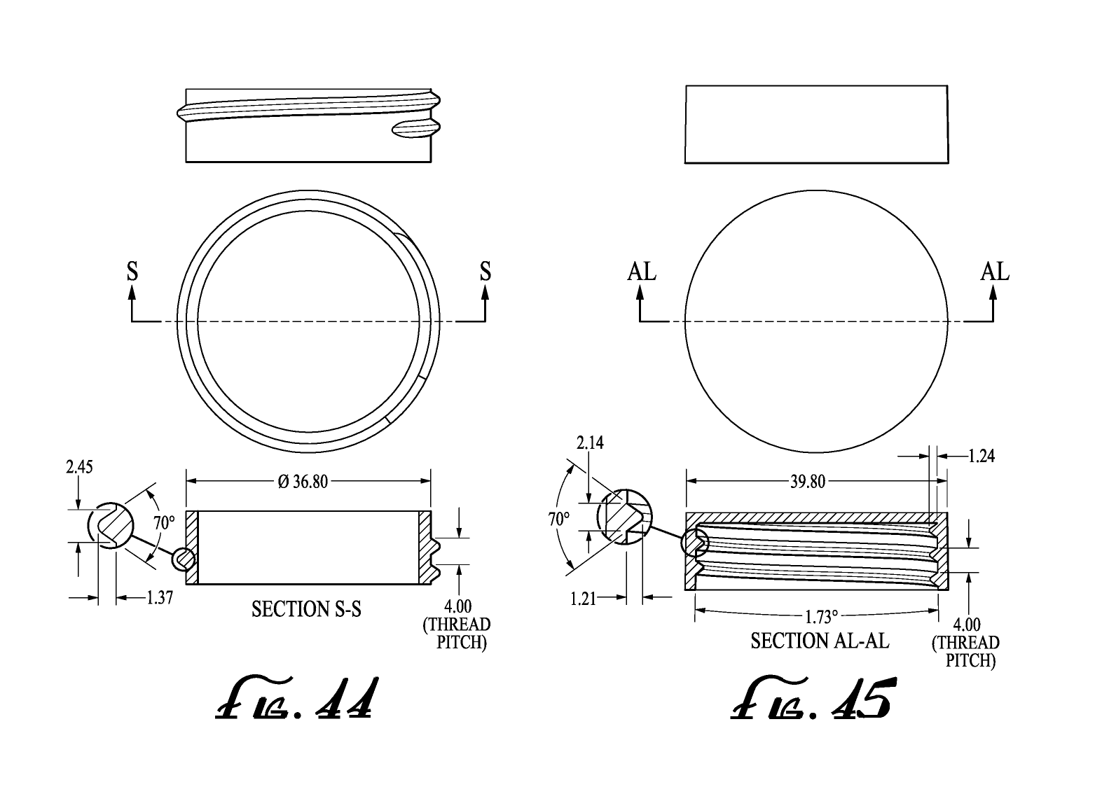

With reference now to the drawing figures, wherein like reference numbers represent corresponding parts throughout the several views, FIGS. 1-43 show various embodiments and aspects of colostrum collection systems 10, 10′ according to example forms of the present invention. In its various forms, the system includes a breast pump 20, 20′ or other means for expressing and collecting colostrum from the breast of a human or other mammal, the breast pump being secured to a syringe collection, containment and delivery device 30 using a collar adaptor 50.

In depicted embodiments, the breast pump 20 includes a collar 22 with a downward depending rim, the interior of which may contain internal screw threading (not pictured). The breast pump collar 22 is ordinarily the coupling site for correspondingly threaded bottles or other containers for collection of breast milk using the pump. In use with the system and method of the present invention, the breast pump collar 22 is adapted for coupling to alternate containers such as a syringe body with the syringe plunger removed, to collect, store and deliver colostrum for feeding from the syringe to a neonate. The syringe 30 typically has an elongated barrel or syringe body 32 which includes an internal chamber defining a contained volume for receiving and discharging fluid contents, a first or distal end that may include a syringe discharge or dispensing port 34, and a second or proximal end which includes an open end 36 defining an opening for receiving the syringe plunger, and a flange 38.

The collar adaptor 50 is generally cylindrical and includes an upper attachment portion 52 configured for detachably coupling with the breast pump collar 22. The collar adaptor 50 also includes a lower base 54 which is attached concentrically with the upper attachment portion 52 and has an outer circumference at least as large as the outer circumference of the breast pump collar 22. The collar adaptor 50 includes a hole 56 extending from center of the top of the attachment portion 52 through the bottom of the base 54.

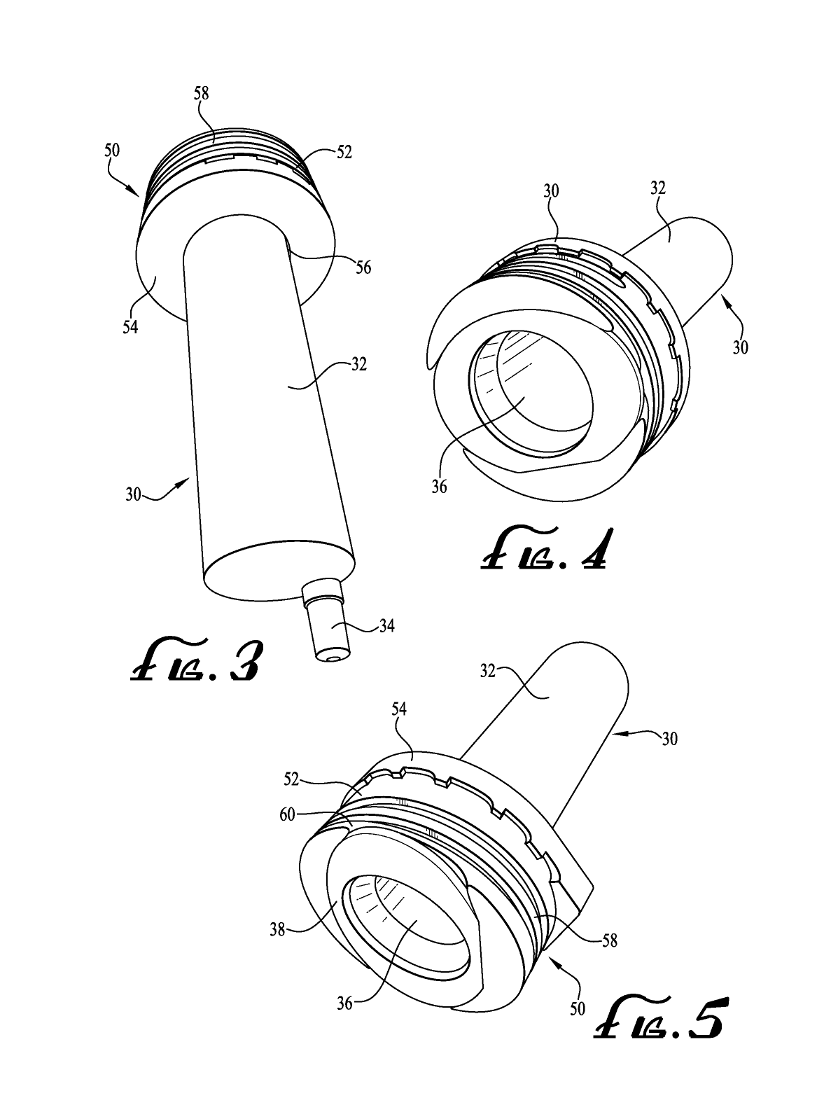

FIGS. 3 and 4 show a collar adaptor 50 according to an example embodiment of the present invention, with a syringe 30 held in the collar adaptor. As depicted, the collar adaptor hole 56 has a size and shape to accommodate the elongated body 32 of the syringe 30. A segment of the attachment portion 52 of the collar adaptor 50 includes external threads 58 to complement the internal threads in the breast pump collar 22. In example embodiments, the threads are a Gerber-style or universal thread compatible with various breast pump makes and models. For example, FIG. 44 shows example details of a male external threaded coupling profile of a collar adaptor, having a minor thread diameter of about 36.80 mm, a thread pitch of about 4.00 mm, a thread angle of about 70°, a thread height of about 1.37 mm, and a thread width of about 2.45 mm. FIG. 45 shows example details of a corresponding female internal thread configuration of an example breast pump collar to which a collar adaptor of a colostrum collection system can be used in connection with, having a nominal major thread diameter of about 39.8 mm, a thread pitch of about 4.00 mm, a thread angle of about 70°, a thread height of about 1.21-1.24 mm, a thread width of about 2.14 mm, and a thread pitch angle of about 1.73°. In alternate embodiments, different thread formats and dimensions can be provided for compatibility with different breast pumps or other equipment. Also, the male and female configurations of the collar adaptor and the breast pump collar can be reversed in alternate embodiments. The top surface of the attachment portion 52 includes a recessed area 60 of size and shape to receive the flange 38 of the syringe 30. In example embodiments, the collar adaptor 50 is constructed of polypropylene, or alternatively of other polymers or other materials.

In the present embodiment, the colostrum collection system 10, 10′ is assembled by inserting the distal end of the syringe 30 downward through the hole 56 of the collar adaptor 50 from the top of the attachment portion 52, through the base 54 until the syringe 30 is positioned with its flange 38 engaged within the recessed area 60 in the top of the collar adaptor 50, and the top of the syringe 30 is flush with the top of the collar adaptor 50. The attachment portion 52 of the collar adaptor 50 is then screwed into the breast pump collar 22, retaining the flange 38 of the syringe 30 between the collar adaptor 50 and the breast pump collar 22. In alternate embodiments, the attachment portion 52 of the collar adaptor 50 is engaged with the breast pump collar 22 by snap couplings, friction fit, threads, interengaging connectors or other retention means.

The breast pump 20 is operated in its intended fashion to express colostrum. The expressed colostrum is directly transferred from the discharge of the breast pump 20 into the syringe 30. After collection, the syringe 30 is removed from engagement with the breast pump 20 and may be stored for subsequent use. A syringe plunger (not pictured) may be inserted into the open end 36 of the elongated body 32 of the syringe 30 in typical fashion for delivery of colostrum from the syringe. The collar adaptor 50 may be removed from the syringe 30 or may remain in place. The colostrum may be delivered directly from the syringe 30 through the dispensing port 32 for feeding to a newborn or neonate via a feeding tube, a nipple attachment, and/or other delivery means connected to the dispensing port 32, for example by advancing the syringe plunger into the syringe 30, by suction through the dispensing port 32, by gravity feed, by dispensing pump, or otherwise. Alternatively, the colostrum can be transferred from the syringe 30 to another container for delivery. Because the sealing head of the syringe plunger wipes down the internal walls of the elongated body 32 of the syringe 30 as it is advanced to dispense the colostrum, fluid loss due to sticking to the container walls is minimized or eliminated. The collar adaptor 50 optionally includes one or more flats or projections along its side surface to prevent it from rolling off of a support surface.

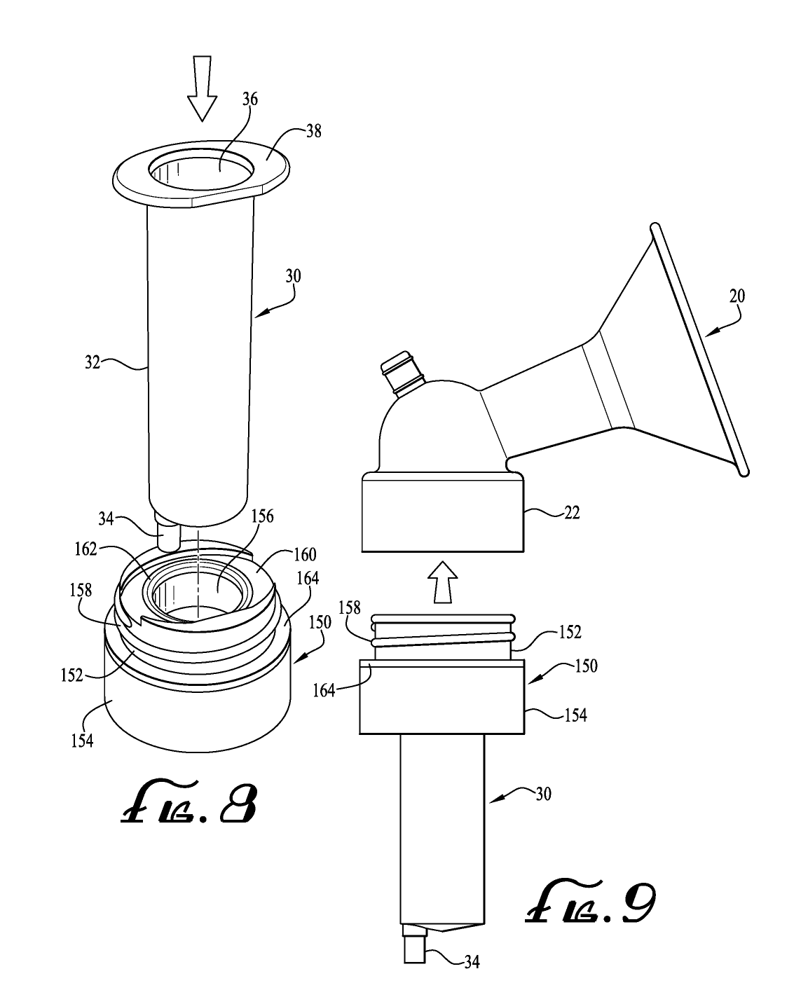

FIGS. 7-10 show a collar adaptor 150 according to another example embodiment of the present invention. As depicted, the collar adaptor 150 includes two ring shaped seal rings or gaskets 162, 164 for providing a liquid tight seal between interfacing elements of the colostrum collection system 10. The adaptor-to-syringe gasket 162 has a width and diameter configured such that the outer surface of the adaptor-to-syringe gasket 162 fits within the collar adaptor hole 156 and the inner surface of the adaptor-to-syringe gasket 162 fits around the elongated body 32 of the syringe 30. The adaptor-to-collar gasket 164 is configured with a width and diameter such that the inner surface of the adaptor-to-collar gasket 164 fits around the attachment portion 152 of the collar adaptor 150 and the outer surface of the adaptor-to-collar gasket 164 does not extend wider than the base 154 of the collar adaptor 150. In example forms, the gaskets 162, 164 can be constructed from various compressible materials, such as rubber, plastic, polymer or other materials of construction suitable for a liquid tight seal.

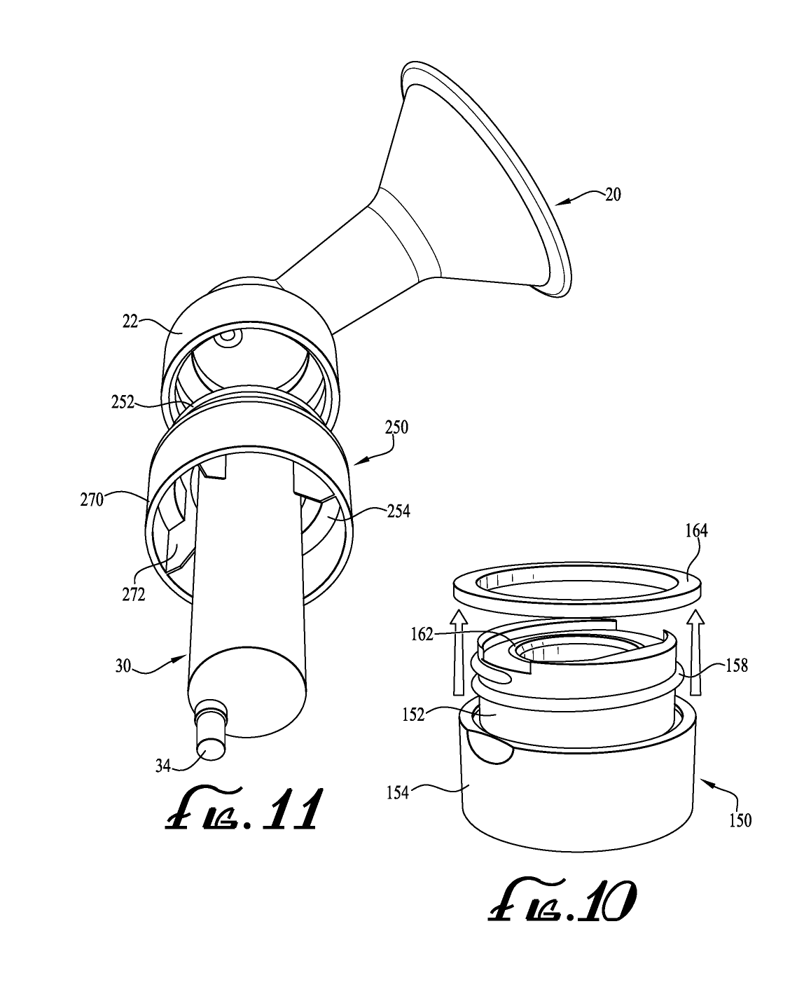

To assemble the system, the adaptor-to-syringe gasket 162 is positioned around the syringe-receiving channel or hole 156 and within the recessed area 160 at the top of the collar adaptor 150 so that when the syringe 30 is moved downward through the collar adaptor 150, as depicted in FIG. 8, it also passes through the adaptor-to-syringe gasket 162. The adaptor-to-syringe gasket 162 is held between the syringe flange 38 and the recessed area 154 of the attachment portion, forming a fluid-tight seal between the syringe 30 and the collar adaptor 150. The adaptor-to-collar gasket 162 is positioned around the attachment portion 152 of the collar adaptor 150 resting against the top of the collar adaptor base 154. When the collar adaptor 150 is screwed into the breast pump collar 22, as depicted in FIG. 9, the adaptor-to-collar gasket 164 is held between the top of the collar adaptor base 154 and the bottom rim of the breast pump collar 22, forming a fluid-tight seal between the collar adaptor 150 and the breast pump collar 22. As depicted in FIG. 10, the adaptor-to-collar gasket 164 is optionally removable from the collar adaptor 150. Installation of the gaskets 162, 164 will be advantageous for use with “open system” breast pumps 20 that utilize a sealed system to maintain efficient pumping action, whereas removal of the gaskets will facilitate use in connection with “closed system” breast pumps 20 to provide venting. Thus, the provision of removable gaskets allows compatibility with both open and closed breast pump systems.

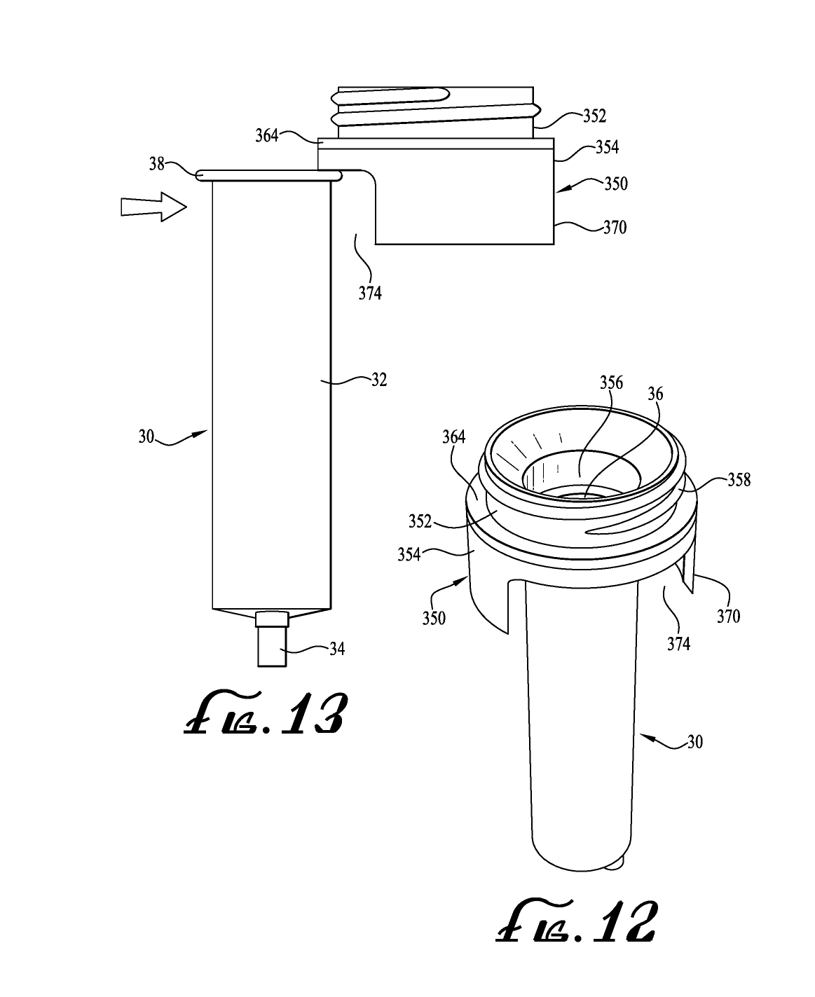

FIG. 11 shows a collar adaptor 250 according to another example embodiment of the present invention. As depicted, the collar adaptor 250 includes a grip sleeve 270 extending downward from the outer circumference of the collar adaptor base 254. The grip sleeve 270 contains a plurality of internal ribs 272. The internal ribs 272 extend inwardly from the inner surface of the grip sleeve 270 towards the center point of the collar adaptor 250 parallel to the bottom surface of the base 254. The internal ribs 272 have a length such that the elongated body of the syringe 32 can be accommodated between the edges of the internal ribs 270 as depicted. The internal ribs 272 strengthen the attachment and aid the fitting of the syringe 30 to the attachment. Optionally, the grip sleeve 270, may have a textured surface to facilitate attaching and detaching the collar adaptor 250 to the breast pump collar 22.

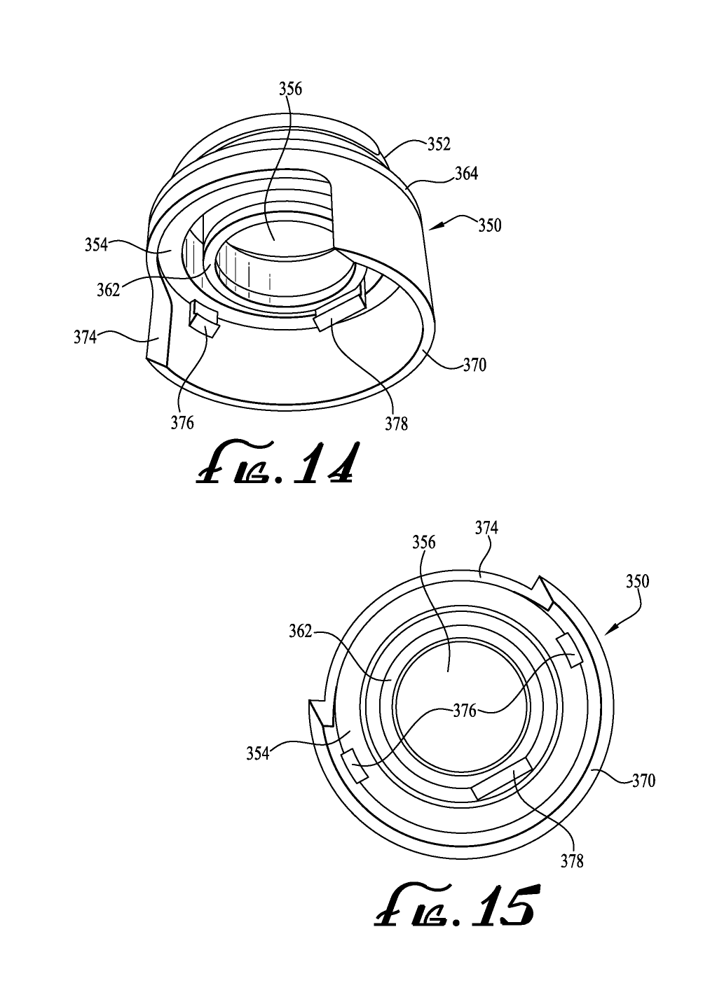

FIGS. 12-15 show a collar adaptor 350 according to another example embodiment of the present invention. As depicted in FIG. 12, the top surface of the attachment portion 352 of the collar adaptor 350 slopes downward from outer edge of attachment portion 352 to the outer surface of the hole 356. The grip sleeve 370 includes a slot 374 that extends vertically from the bottom of the grip sleeve 370 to a distance below the collar adaptor base 354, and horizontally a dimension configured to accommodate the length of the syringe flange 38. As depicted in FIGS. 14 and 15, the bottom of the collar adaptor base, within the grip sleeve 370, contains two end clips 376 which are perpendicular to the length of the slot 374, and one side clip 378, which is parallel to the length of the slot 374. The clips 376, 378 are long enough to accommodate the syringe flange 38. The end clips 376 are positioned a distance apart such that they will engage with the ends of the length of the syringe flange 38 when it is positioned with the open end 36 of the syringe 30 below the collar adaptor hole 356. The side clip 378 is positioned to engage with the side of the flange 38 when the syringe 30 is in the same position. An adaptor-to-syringe gasket 362, with an inner diameter approximately equal to the diameter of the inner surface of the syringe elongated body 32 is attached to the bottom of the collar adaptor base 354 so that the inner surface of the adaptor-to-syringe gasket 362 is aligned with the inner surface of the collar adaptor hole 356. The clips are optionally configured to receive and engage the syringe flange with a friction or interference fit, and/or are sufficiently flexible to resiliently engage the syringe flange. The syringe 30 is removably engaged with the collar adaptor 350 by positioning the syringe flange 38 so that its length is parallel with the length of the slot 374 in the grip sleeve 370. As depicted in FIG. 13, the syringe 30 slides laterally into the grip sleeve 370 and is positioned so that the syringe flange 38 engages with the clips 376, 378. The end clips 376 engage with the ends of the flange 38 length and the side clip 378 engages with one side of the flange 38 width. The clips 376, 378 secure the syringe 30 to the bottom of the base 354 of the collar adaptor 350 such that the open end 36 of the syringe 30 is aligned with the collar adaptor hole 356 and the adaptor-to-syringe gasket 362 forms a liquid tight seal between the syringe 30 and the collar adaptor 350. In this example embodiment it is not necessary to detach the collar adaptor 350 from the breast pump collar 22 in order to attach and detach the syringe 30 from the collar adaptor 350. The sloped surface of the attachment portion 352 prevents plugging of the syringe 30 by a breast pump one-way valve. This embodiment can also employ a removable adaptor-to-collar gasket 364 in the same position as previously described.

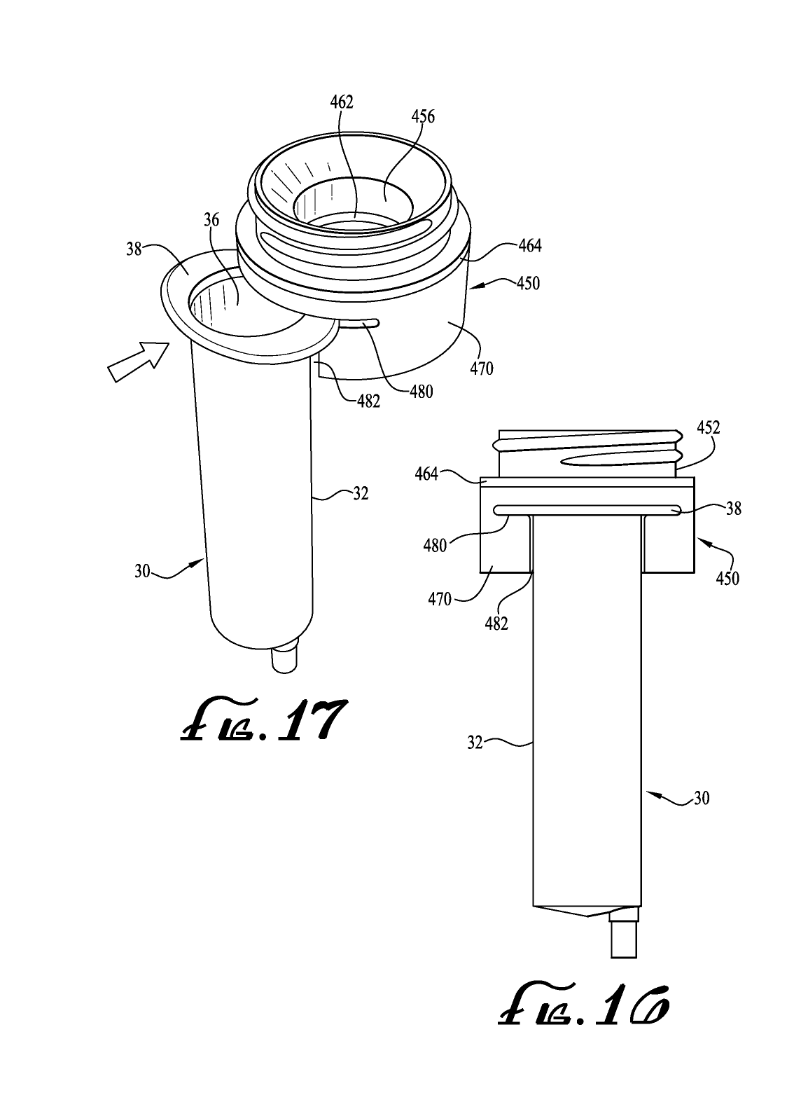

FIGS. 16 and 17 show a collar adaptor 450 according to another example embodiment of the present invention. As depicted, the collar adaptor comprises a grip sleeve 470 including a slotted opening or slit 480 which is parallel to the bottom of the collar adaptor base 454. The slit 480 has a length and width configured to slidingly receive the flange 38 of the syringe 30 therein. The grip sleeve 470 also includes a channel or slot 482 extending downward from the center of the slit 480 to the bottom of the grip sleeve 470, the width of which is such that it can accommodate elongated body 32 of the syringe 30. The collar adaptor 450 in FIGS. 16 and 17 may include clips 476, 478 and gaskets 462, 464 in similar fashion to those features depicted in FIGS. 14 and 15 and described above. The syringe 30 is engaged with the collar adaptor 450 by positioning the length of the syringe flange 38 parallel to and aligned with the slit 480 in the grip sleeve 470 and sliding the syringe flange into the slit. As depicted in FIG. 17, the syringe 30 slides laterally into the collar adaptor such that the syringe flange 38 passes through the slit 480 and the syringe elongated body 32 passes through the slot 482 in the grip sleeve 470. The syringe 30 can be retained in engagement with the collar adaptor base 454 using clips 476, 478 as described above, by friction engagement or a close tolerance fit between the syringe flange 38 and the slotted opening 480, or by other retention means. The syringe 30 is removed by reversing the process to slide the syringe flange 38 out of the slotted opening 480 of the collar adaptor 450.

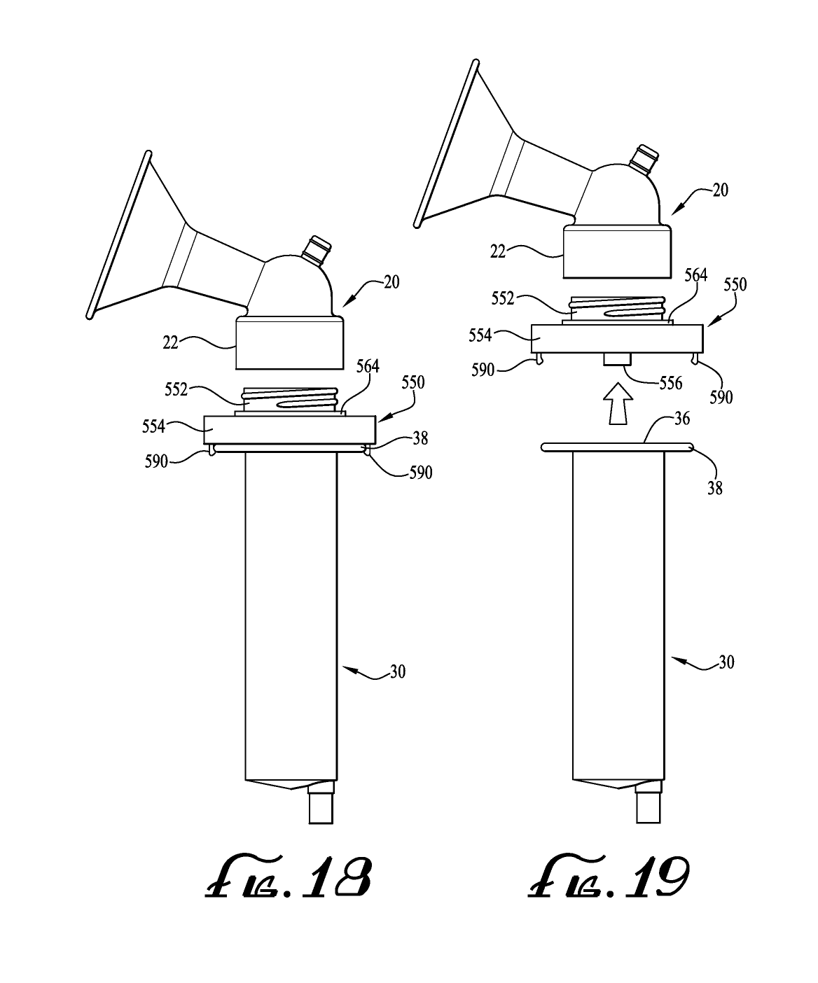

FIGS. 18 and 19 show a collar adaptor 550 according to another example embodiment of the same invention. As depicted, a plurality of clips 590 protrude from bottom of the collar adaptor base 554. The clips 590 are of sufficient length to accommodate the thickness of the syringe flange 38. The clips 590 are positioned where they will engage with the syringe flange 38 when it is positioned such that the syringe opening 36 is aligned with the collar adaptor hole 556. As depicted in FIG. 19, the top of the syringe 30 is press fitted to the bottom base 554 of the collar adaptor 30 so that the clips 590 engage with the syringe flange 38, and resiliently snap over the edges of the flange to removably secure the top of the syringe 30 to the bottom of collar adaptor 550. The attachment portion 552 of the collar adaptor 550 includes a threaded fitting to be screwed into breast pump collar 22. This embodiment optionally enables the collar adaptor 550 to accommodate a larger syringe 30 with a wider flange 38. It also allows different size syringes 30 to be fitted to the same collar adaptor 550 because this embodiment removes the constraints of working inside the dimensions of the threaded connection.

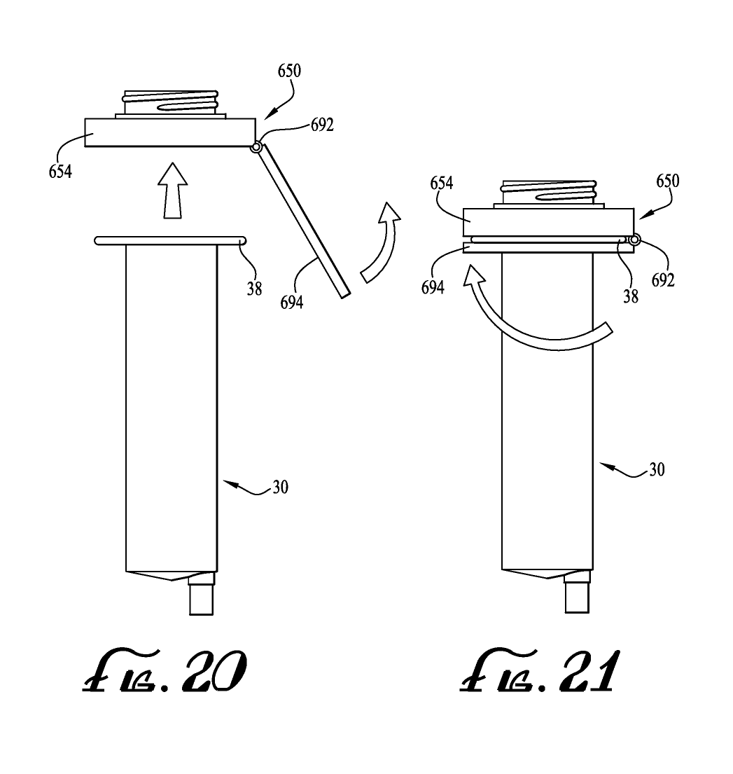

FIGS. 20 and 21 show a collar adaptor 650 according to another example embodiment of the same invention. As depicted, the collar adaptor base 654 includes a hinge 692 and pivotally mounted retention arm 694. In use, the syringe flange 38 is secured between the retention arm 694 and the collar adaptor base 654 which keeps the syringe 30 attached to the collar adaptor 650. A releasable snap coupling, latch or other closure means may be provided to hold the retention arm 694 closed with the syringe flange 38 engaged with the collar adaptor 650. Optionally, one or more clips 690 as depicted in FIGS. 18 and 19 and described above may be used in conjunction with the hinge 692 and arm 694. The collar adaptor 650 can be secured to the breast pump 20 by threaded coupling in the manner described above.

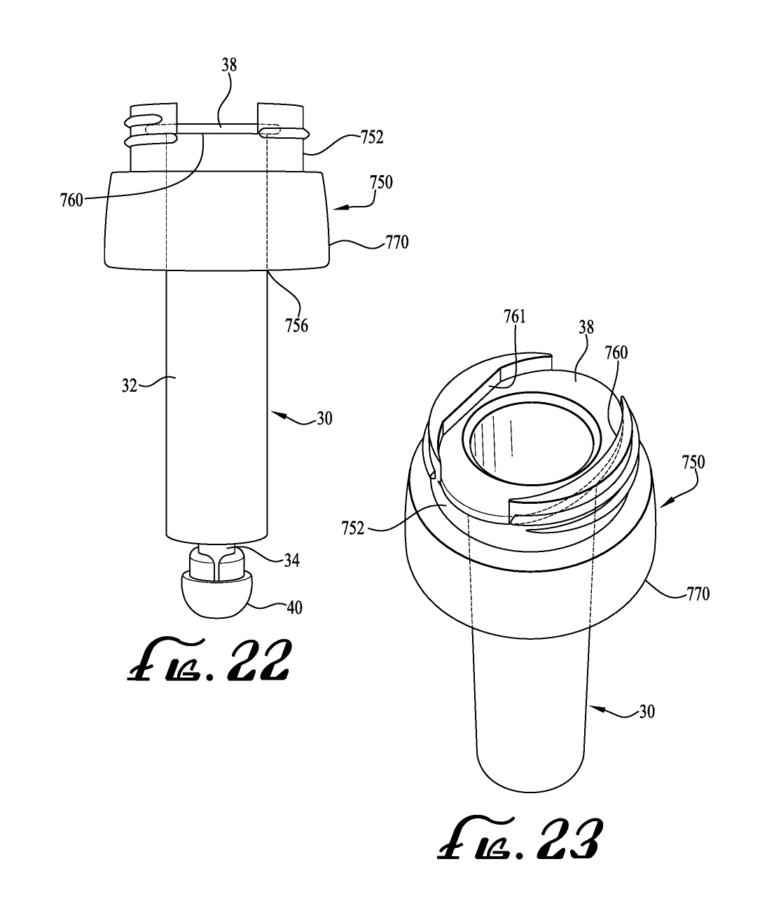

FIGS. 22 and 23 show a collar adaptor 750 according to another example embodiment of the same invention. As depicted, the top of the attachment portion 752 of the collar adaptor 750 includes a recessed area 760 approximately the size and shape of the syringe flange 38, preferably slightly larger so that the syringe flange 38 easily fits in the recessed area 760. The recessed area 760 has a depth such that when the bottom of the syringe flange 38 is resting on the bottom of the recessed area 760, the top of the syringe flange 38 is below the top of the attachment portion 752 of the collar adaptor 750. The attachment portion 752 may include a protrusion 761 that extends from one side wall of the recessed area 760, beginning a distance above the bottom of the recessed area 760 large enough to accommodate the thickness of the syringe flange 38. As depicted, the protrusion 761 extends vertically to the top of the attachment portion 752. The grip sleeve 770 defines a tapered or bowed profile, where its diameter increases from the top to the bottom. The syringe 30 is inserted into the collar adaptor 750 by inserting the distal end of the syringe 30 downward through the collar adaptor hole 756 from the top of the attachment portion 752 until the syringe 30 is positioned such that the flange 38 is received in the recessed area and against the protrusion 761. The syringe 30 is held such that the bottom of the syringe flange 38 rests on the bottom of the recessed area 760, and the top of the syringe 30 sits below the top of the collar adaptor 750. In this embodiment, the recessed seated position of the syringe 30 within the attachment portion 752 of the collar adaptor 750 facilitates threading the attachment portion 752 into the breast pump collar 22. The seated position of the syringe 30 also provides clearance for a one-way valve (not pictured) that may be present on the breast pump 20. The tapered or bowed profile of the grip sleeve 770 allows a user to more readily differentiate between the collar attachment 750 and the breast pump collar 22 for removal of the collar from the pump. The syringe tip or discharge port 34 at the distal end of the syringe body 32 can be closed by a removable tip cap 40 as shown, or by a plug or other closure, to prevent unintended discharge of fluid from the syringe 30 during collection and storage. U.S. patent application Ser. No. 14/844,922 is incorporated by reference as showing an example tip cap 40 suitable for use on connection with the syringe 30.

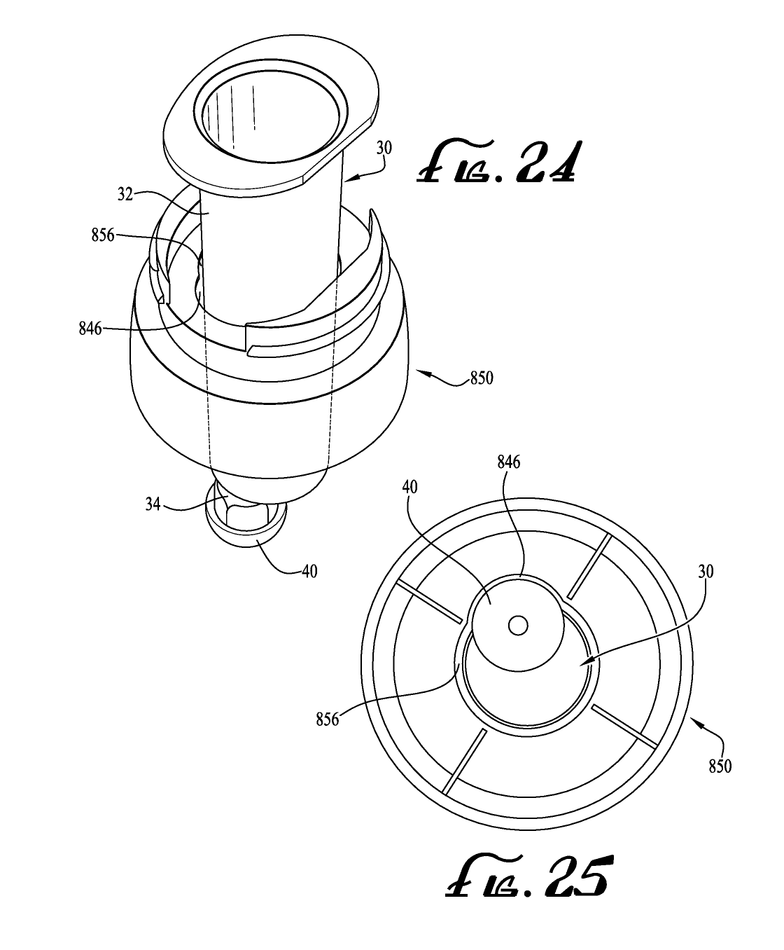

FIGS. 24-26 show a collar adaptor 850 according to another example embodiment of the same invention. As depicted, the dispensing port 34 of the syringe 30 includes a removable tip cap 40. The tip cap 40 is a size where, when attached to the dispensing port 34, a portion of the tip cap 40 extends beyond the outer surface of the syringe elongated body 32. As depicted in FIG. 25, the hole 856′ in the collar adaptor 850 includes a rounded cutout 846, the diameter of which is large enough to accommodate tip cap 40 when it is attached to the dispensing port 34. The syringe 30 is inserted into the collar adaptor by aligning the attached tip cap 40 with the cutout 846 in the collar adaptor hole 856′ and moving the distal end of the syringe 30 downward through the hole 856′ of the collar adaptor 850 from the top of the attachment portion 852.

FIG. 27-30 show a removable gasket 962 for use with a collar adaptor 950 such as shown in FIGS. 22-26. As depicted, the gasket 962 comprises two gasket subcomponent seal portions 964, 966. The upper portion 964 is shaped to fit the lower surface of the recessed area 960 in the attachment portion 952 of the collar adaptor 950. The lower portion 966 has generally the same shape and size as the previously described adaptor-to-collar gasket. The two gasket portions 964, 966 are formed as one unitary gasket 962 using one or more connecting members or webs 968. The connecting members position the upper portion 964 of the gasket 962 such that it rests on the bottom surface of the recessed area 960 and the inner surface is aligned with the collar adaptor hole 956, and the lower portion 966 rests on the top of the collar adaptor base 954. Typically, the gasket 962 is formed of a compressible material for forming a fluid tight seal between components. As depicted in FIG. 27, the gasket 962 is press fitted to the collar adaptor 950 and is easily removable. The gasket 962 is positioned so that the shape of the upper portion 964 is aligned with the bottom surface of the recessed area 960 and the inner surface of the upper portion 964 of the gasket 962 is aligned with the collar adaptor hole 956. As depicted in FIG. 30, the syringe 30 is inserted into the collar adaptor 950 by moving the distal end of the syringe 30 downward through the hole 956 and the upper portion 964 of the gasket 962. The upper portion 964 of the gasket 962 is held between the collar adaptor 950 and the syringe flange 38, forming a liquid tight seal. When the collar adaptor 950 is attached to the breast pump collar 22, the lower portion 966 of the gasket 962 is held between the collar adaptor base 954 and the rim of the breast pump collar 22, forming a liquid tight seal.

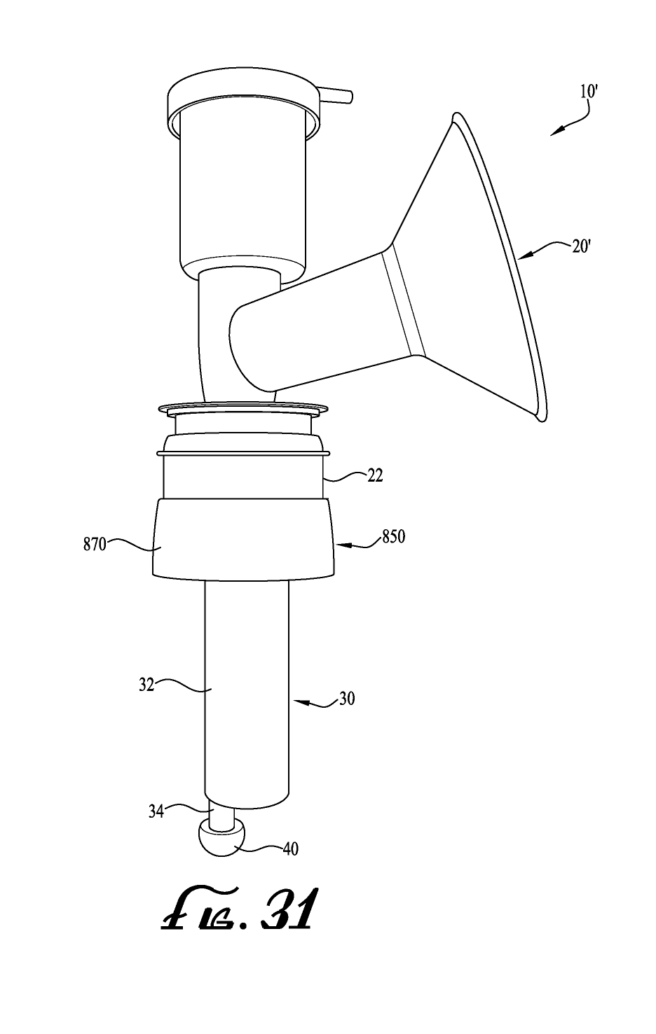

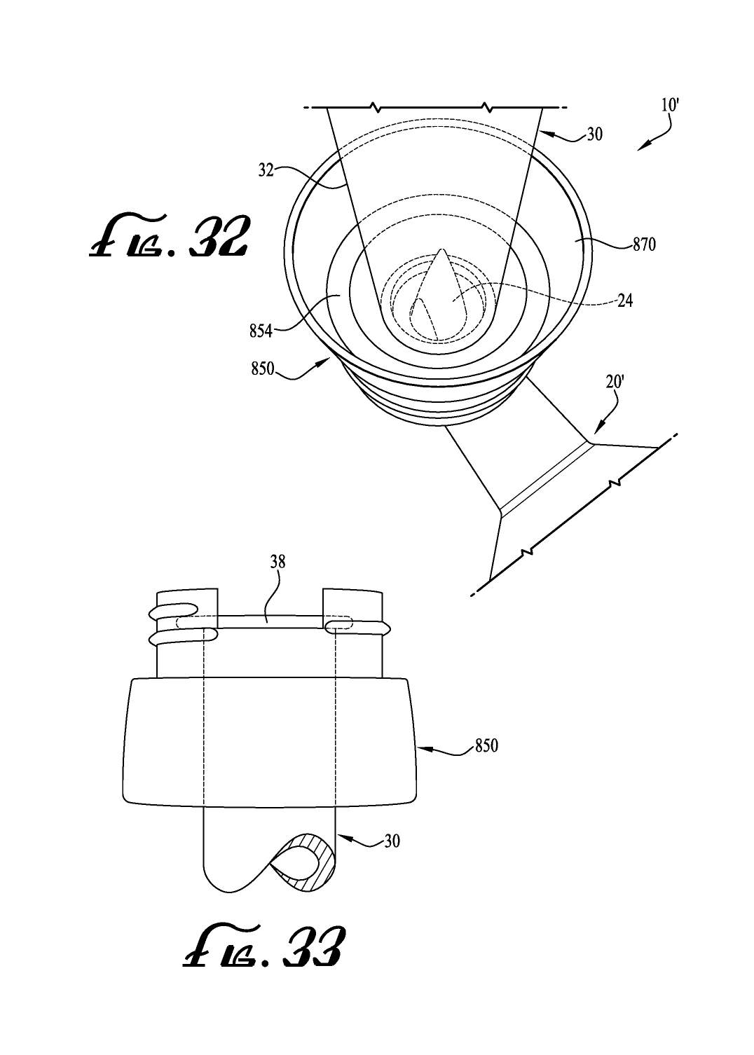

FIGS. 31-37 show the collar adaptor 850 of FIGS. 24-26 attached to various different types of commercially available breast pumps 20, 20′. As depicted in FIGS. 31-33, the collar adaptor 850 and syringe 30 are fitted to an Ameda Purely Yours breast pump 20′. As described above, the seated position of the flange 38 of the syringe 30, depicted in FIG. 33, creates clearance which prevents the Ameda one-way valve 24, depicted in FIG. 32, from occluding the syringe elongated body 32 and creating a vacuum. As depicted in FIGS. 34-37, the collar adaptor 850 and syringe 30 are fitted with a Limerick breast pump 20. In example forms, the collar adaptor comprises a universal fit coupling configuration for use with a plurality of different breast pump types and brands, and/or one or more adaptor couplings are provided to allow connection to different breast pump types and brands.

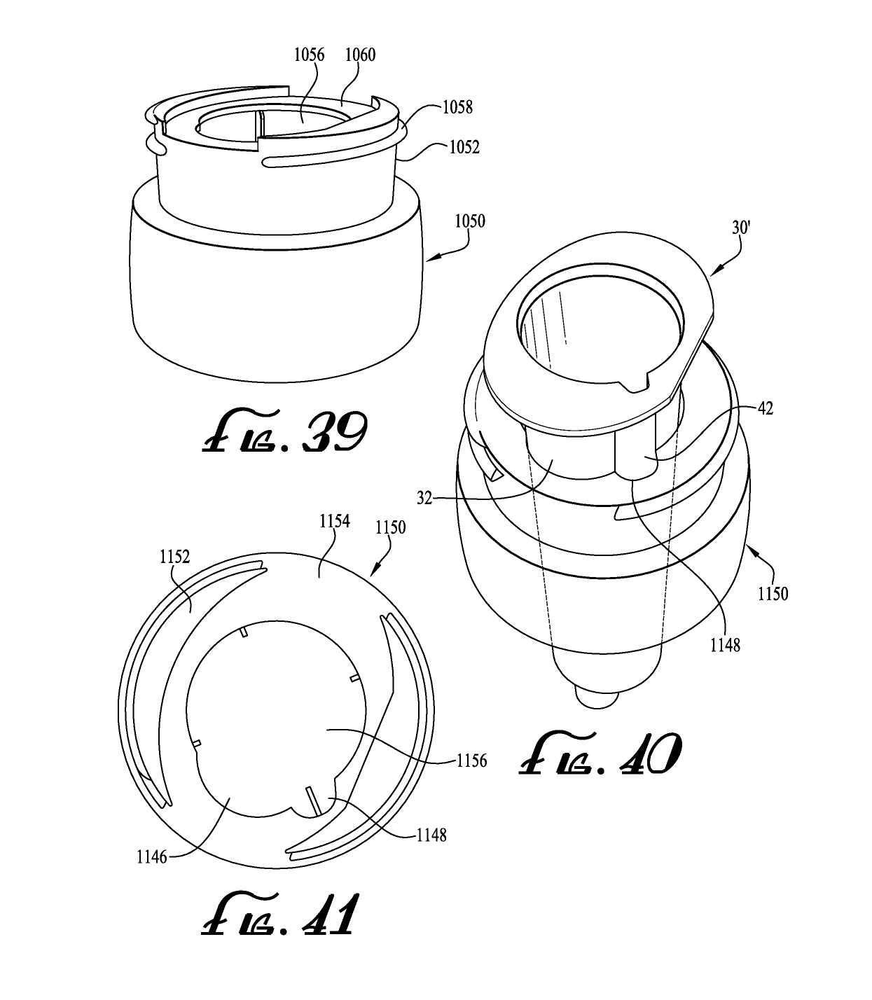

FIGS. 38 and 39 show a collar adaptor 1050 according to another example embodiment of the present invention. As depicted, the top of the attachment portion 1052 of the collar adaptor 1050 includes a recessed area 1060, the depth of which is approximately equal to the thickness of the syringe flange 38. The attachment portion 1052 includes external threads 1058 which begin at the top surface of the attachment portion 1052. When the syringe 30 is inserted into the collar adaptor 1050, the flange 38 is positioned within the recessed area 1060 so that the top of the syringe 30 is flush with the top of the attachment portion 1052 of the collar adaptor 1050. A gasket (not pictured) may be provided to sit atop the top surface of the collar adaptor 1050 once the syringe 30 is within the hole 1056′ and the flange 38 is seated within the recessed area 1060.

FIGS. 40-43 show a collar adaptor 1150 according to another example embodiment of the present invention. As depicted, the hole 1156 in the collar adaptor 1150 includes a notch 1148 of size and shape to provide clearance for a vent 42 that may be present on the syringe 30′. In example forms, the vent 42 is in the form of an indent or rib formed on an outer periphery of the syringe elongated body 32. U.S. Pat. No. 8,992,448, which is herby incorporated by reference herein, discloses a vented syringe to which the present invention may be adapted. Optionally, two or more notches 1148 may be provided for syringes 30′ comprising two or more vents 42 as depicted in FIG. 42. Optionally, the clearance cutout 1146 provided for the tip cap 40 may be combined with the clearance notch 1148 provided for the vent 42, as depicted in FIG. 40, in the event a syringe 30 contains both a vent 42 and removable tip cap 40.

While the depicted embodiments generally depict examples having external threading 58 on the collar adaptor 50 to attach the collar adaptor 50 to internal threading on the breast pump collar 22, in alternate embodiments, a snap-fit coupling, friction fit or other attachment means may be utilized to engage the collar adaptor 50 with the breast pump 22. While the collar adaptor 50 has been described as cylindrical, it may have any alternate size, shape, contour and overall design so long as it is compatible with the collar 22 or attachment portion of the breast pump 20.

In example embodiments, the syringe 30 utilized in connection with the system and method of the present invention comprises a 20 ml NeoMed syringe. However, other sizes and/or brands of syringes 30 may be used with different collar adaptor 50 designs or the collar adaptor 50 may take a universal fit form that would allow for compatibility with multiple syringe 30 sizes and formats. The collar adaptor 50 may be configured for compatibility with various syringe flange 38 designs or the collar adaptor 50 may take a universal fit form that would allow for compatibility with multiple different syringe flange 38 designs.

In example embodiments, open system breast pumps 20 may be used in connection with the colostrum collection system 10, such as for example, Medela (Pump in Style, Pump in Style Advanced, Pump in Style Advanced Backpack, Symphony), Evenflo (Comfort Select Dual, Elan Single, Elan Dual), First Years (Natural Comfort Double, Easy Comfort, miPump Deluxe Double, miPump Double, miPump Single). In other example embodiments, closed system breast pumps 20 may be used, such as for example, Avent ISIS iQ Duo, Ameda (Elite, Purely Yours, Purely Yours w/carryall, Purely Yours w/backpack), Playtex Embrace, Nailey Medical Nurture III, Evenflo Comfort select, First Years Natural Comfort Double, Lansinoh Double Electric, Avent ISIS iQ Uno, Avent ISIS iQ Uno Complete, First Years Natural Comfort Single, Medela Swing, and Hygeia (EnDeare, Enjoye-LBI, Enjoye-EXT, Enjoye-EPS). In further alternate embodiments, other breast pumps 20 (open, closed, or otherwise) may be adapted for use in connection with the colostrum collection system 10 according to example forms of the invention.

In example methods of use, the system of the present invention enables the collection of colostrum by providing a collar adaptor having a receiver for engaging the proximal end or flange of a syringe body, and a coupling for attachment to the discharge of a breast pump, engaging the proximal end or flange of the syringe body with the receiver of the collar adaptor, attaching the coupling of the collar adaptor to the breast pump, and operating the breast pump to express colostrum from the breast and deliver the colostrum directly into the internal contained volume of the syringe. The method optionally further comprises removing the syringe from the collar adaptor, capping the distal discharge port of the syringe, inserting the syringe plunger or other closure means into the proximal opening of the syringe body to close the syringe for storage, removal of the cap from the discharge port, attachment of a feeding tube or other fluid transfer means to the discharge port, and/or enteral feeding delivery of colostrum from the syringe by advancing the syringe plunger into the syringe body. The method optionally further comprises addition of one or more supplements, medications, formula, breast milk or other constituents to the collected colostrum for enteral delivery. Also, while the embodiments are generally described herein for use in collecting and dispensing colostrum, the disclosed systems and components may be used for collecting and dispensing breast milk, formula, medications, supplements, and/or other enteral fluids.

While the invention has been described with reference to preferred and example embodiments, it will be understood by those skilled in the art that a variety of modifications, additions and deletions are within the scope of the invention, as defined by the following claims.

What is claimed is:

1. A system for collection of colostrum, the system comprising:
a breast pump for expressing colostrum from a mammalian breast, the breast pump having a breast pump discharge port for delivery of the expressed colostrum;
a collection syringe body comprising an internal chamber, a proximal end having a flange and a plunger opening in fluid communication with the internal chamber, and a distal end having a syringe discharge port in fluid communication with the internal chamber; and
a collar adaptor comprising a receiver for releasable engagement of the proximal end of the syringe body and a coupling for attachment to the breast pump discharge port.

2. The system of claim 1, wherein the collar adaptor retains the plunger opening of the syringe body in direct fluid communication with the breast pump discharge port to directly transfer the expressed colostrum from the breast pump discharge port through the plunger opening and into the internal chamber of the syringe body.

3. The system of claim 1, wherein the receiver of the collar adaptor comprises a recessed area formed within the collar adaptor, the recessed area configured to receive the flange of the syringe body.

4. The system of claim 1, wherein the receiver of the collar adaptor comprises a plurality of resilient clips for engaging the flange of the syringe body.

5. The system of claim 1, wherein the receiver of the collar adaptor comprises a slotted opening configured to slidingly receive the flange of the syringe body.

6. The system of claim 1, wherein the receiver of the collar adaptor comprises a pivotal retention arm for engaging the flange of the syringe body.

7. The system of claim 1, further comprising a tip cap for closure of the syringe discharge port.

8. The system of claim 1, further comprising a syringe plunger configured to be received through the plunger opening of the syringe body and advanced and retracted within the internal chamber of the syringe body.

9. The system of claim 1, wherein the collar adaptor further comprises at least one gasket for sealing a fluid path between the breast pump and the collection syringe body.

10. The system of claim 1, wherein the collar adaptor coupling comprises a threaded coupling cooperative with a corresponding breast pump coupling adjacent the breast pump discharge port.

11. The system of claim 1, wherein the collar adaptor coupling is universally compatible with a plurality of breast pump types.

12. The system of claim 1, wherein the collar adaptor further comprises an inwardly sloped inlet.

13. The system of claim 1, wherein the collar adaptor comprises a channel extending therethrough for passage of the syringe body.

14. The system of claim 13, wherein the channel through the collar adaptor comprises a recess for clearance of a syringe tip cap installed on the syringe discharge port of the syringe body.

15. The system of claim 13, wherein the channel through the collar adaptor comprises a recess for clearance of a syringe vent formed on the syringe body.

16. A collar adaptor for a colostrum collection system, the collar adaptor comprising a receiver for releasable engagement of a proximal end of a syringe body and a coupling for attachment to a breast pump discharge port.

17. The collar adaptor of claim 16, further comprising retention means for retaining a plunger opening of the syringe body in direct fluid communication with the breast pump discharge port to directly transfer expressed colostrum from the breast pump discharge port through the plunger opening and into an internal chamber of the syringe body.

18. The collar adaptor of claim 16, wherein the retention means comprises a recessed area formed within the collar adaptor, the recessed area configured to receive a flange of the syringe body.

19. The collar adaptor of claim 16, wherein the retention means comprises a plurality of resilient clips for engaging a flange of the syringe body.

20. The collar adaptor of claim 16, wherein the retention means comprises a slotted opening configured to slidingly receive a flange of the syringe body.

21. The collar adaptor of claim 16, wherein the retention means comprises a pivotal retention arm for engaging a flange of the syringe body.

22. The collar adaptor of claim 16, further comprising at least one gasket for sealing a fluid path between the breast pump and the syringe body.

23. The collar adaptor of claim 16, further comprising a threaded coupling cooperative with a corresponding breast pump coupling adjacent the breast pump discharge port.

24. The collar adaptor of claim 23, wherein the threaded coupling is universally compatible with a plurality of breast pump types.

25. The collar adaptor of claim 16, further comprising an inwardly sloped inlet adjacent the coupling for attachment to the breast pump discharge port.

26. The collar adaptor of claim 16, further comprising a channel extending therethrough for passage of the syringe body.

27. The collar adaptor of claim 26, wherein the channel extending therethrough comprises a recess for clearance of a syringe tip cap installed on the syringe body.

28. The collar adaptor of claim 26, wherein the channel extending therethrough comprises a recess for clearance of a syringe vent on the syringe body.

29. A method of collecting colostrum from a mammalian breast into a containment syringe, the method comprising:
coupling a collection syringe body to a breast pump with a collar adaptor, the collection syringe body comprising an internal chamber for receiving and containing collected colostrum, a proximal end having a flange and a plunger opening in fluid communication with the internal chamber, and a distal end having a syringe discharge port in fluid communication with the internal chamber; the breast pump comprising a breast pump discharge port; and the collar adaptor comprising a receiver for releasable engagement of the proximal end of the syringe body and a coupling for attachment to the breast pump discharge port;
expressing colostrum from the mammalian breast by operation of the breast pump; and
discharging the expressed colostrum from the breast pump discharge port directly to the internal chamber of the collection syringe body through the plunger opening.
